(19) United States
(12) Patent Application Publication
(10) Pub. No.: US 2020/0275922 A1
(43) Pub. Date: Sep. 3, 2020

(54) SUTURE PASSER AND METHOD OF TISSUE REPAIR

(71) Applicant: Arthrex, Inc., Naples, FL (US)

(72) Inventors: Jason A. Valentin, Fort Myers, FL (US); Keith G. Taylor, Naples, FL (US)

(73) Assignee: Arthrex, Inc., Naples, FL (US)

(21) Appl. No.: 16/288,728

(22) Filed: Feb. 28, 2019

(51) Int. Cl. A61B 17/04; A61L 17/00; A61B 17/06

(52) U.S. Cl. CPC A61B 17/0491; A61L 17/00; A61B 2017/2926; A61B 2017/0477; A61B 17/06066

(57) ABSTRACT

A suture passer and methods for surgical repairs are disclosed. A suture passer can position a first portion of a suturing construct around tissue and simultaneously pass another portion of the suturing construct through the tissue. The suturing construct may be a loop terminating in a single tail.

### BACKGROUND

The disclosure relates to the field of surgery and, more specifically, to suturing instruments and methods of passing suture.

### SUMMARY

A suture passer and methods for surgical repairs are disclosed. A suture passer can pass a first portion of a suturing construct through tissue and another portion of the suturing construct around the tissue. The suturing construct may be a loop terminating in a single tail.

Methods of passing suture are also disclosed. A first portion of a flexible strand is passed through soft tissue while a second portion of the flexible strand is passed around the soft tissue, in one step.

### BRIEF DESCRIPTION OF THE DRAWINGS

FIG. 1 illustrates a cross-sectional view of a suture passing instrument according to an exemplary embodiment.

FIG. 2 illustrates another cross-sectional view of the suture passing instrument of FIG. 1.

FIG. 3 illustrates an enlarged view of the distal end of the suture passing instrument of FIG. 1.

FIG. 4 illustrates a perspective view of the distal end of the suture passing instrument of FIG. 3.

FIG. 5 illustrates an enlarged view of the distal end of the suture passing instrument of FIG. 2.

FIG. 6 illustrates a perspective view of the distal end of the suture passing instrument of FIG. 5.

FIG. 7 is a perspective view of a needle of the suture passing instrument of FIG. 1.

FIG. 8 is an enlarged view of the distal end of the needle of FIG. 8.

FIG. 9 illustrates a flexible construct of the suture passing instrument of FIG. 1.

FIGS. 10-13 illustrate schematic steps of a tissue repair with the suture passing instrument of FIG. 1.

FIG. 14 illustrates a suture passing instrument according to another exemplary embodiment.

FIGS. 15-28 illustrate an exemplary method of tissue repair with the suture passing instrument of FIG. 1.

### DETAILED DESCRIPTION

The disclosure provides a suture passer and techniques for surgical repairs such as endoscopic surgical repairs (for example, arthroscopic surgeries). A suture passer can clamp or secure a tissue in place, pass a needle through the tissue, load a first portion of a flexible construct into the needle, retract the needle with the first portion back through the tissue, and position a second portion of the same flexible construct around the same tissue. The flexible construct may be a suture loop terminating in a single tail. In an embodiment, the tissue can be soil tissue such as tendon or ligament. In an embodiment, the tissue can be a capsule. A suture passer can facilitate suture placement in soft tissue and formation of a Loop ‘N’ Tack™ stitch in one step.

A suture passing instrument can include an elongated tubular member that has a channel housing a needle, and a tip configured to be loaded with a flexible construct in the form of a loop terminating in a single tail. The loop and the single tail are passed independently and without the need to pull the instrument out of the surgical site for reloading. The flexible construct is loaded onto the instrument outside of the surgical site. The loop is loaded onto a jaw and the single tail is loaded onto an opposite jaw. The loop and the single tail are then independently passed inside the surgical site (without the need to pull the instrument out of the surgical site to reload the sutures). A needle captures the single tail and passes the single tail through the loop and through the tissue at the surgical site. The loop surrounds the tissue. The suture passing instrument eliminates multiple instruments needed to perform a stitch, improving OR time and the efficiency of the overall procedure.

The suture passing instrument is first loaded with the required suture which then allows the user to encompass (encapsulate) the soft tissue with the suture. Once the tissue is captured and the sutures are properly positioned to encapsulate the tissue, the needle can be deployed to penetrate the tissue. When the needle is retracted, the needle retrieves the suture and pulls it through the tissue. When the user reduces slack, what is produced is a Loop ‘N’ Tack™ stitch.

A suture passing instrument can include an elongated tubular member that has a channel housing, a needle, and a tip configured to be loaded with a flexible construct in the form of a suture loop terminating in a single tail. The suture passing instrument can include a handle assembly located at the proximal end of the elongated tubular member. The tip is located at the distal end of the elongated tubular member. The tip comprises a fixed lower jaw and an upper movable jaw. The handle assembly can comprise a first trigger mechanism, a second trigger mechanism, and a handle, the first and second trigger mechanisms being connected to the handle. The handle assembly provides for opening and closing of the upper jaw coupled to the tip of the instrument. A ratcheting mechanism may provide for locking and unlocking of the upper jaw as desired by the user. Additionally, the handle assembly houses a mechanism for advancing and retracting the needle.

A trigger mechanism can articulate the upper jaw (movable jaw or movable member) relative to the tip. A loop of the flexible construct can be placed in a securing mechanism (slot or opening) of the upper jaw. A single tail of the flexible construct can be placed in a slot or opening of the lower jaw (stationary jaw or immobile jaw) of the instrument. By squeezing one trigger mechanism, the movable jaw can pivot/move towards the stationary jaw, thereby securing contact with tissue and clamping the tissue between the jaws. By actuating another trigger mechanism, a needle can be advanced relative to a body of the instrument. The needle can be pushed forward in a direction about parallel to the longitudinal axis of the instrument, to capture the single tail extending within the slot in the lower jaw and when pulled back, the single tail (i.e., captured by the needle) passes through the loop and the tissue. Pulling on the single tail allows the loop to surround the tissue.

In an illustrative embodiment, a suture passing instrument comprises two securing mechanisms. A securing mechanism can include a transversal slot or opening, or multiple slots or openings. In an embodiment, a first jaw comprises a distal opening for securing a tail of a flexible construct. A first jaw can be fixedly mounted to a distal end of a shaft. In an embodiment a second jaw is movable in respect to a first jaw. In an embodiment, a second jaw comprises a securing mechanism (a distal opening) for securing a loop of the flexible construct. A suture passing instrument can also include a handle assembly. A handle assembly can move the needle from a first position to a second position, and can also move the second jaw with respect to the first jaw. In an illustrative embodiment, the single tail is captured by and loaded onto the needle (e.g., onto a hook of a needle), thereby passing the single tail of the flexible construct through the tissue and through the loop.

In an embodiment, a suture passer instrument comprises: a shaft having a longitudinal axis, a distal end, and a proximal end; a tip at the distal end of the shaft, the tip having an opening for a needle to pass through and a securing mechanism to allow secure placement of single tail of a flexible construct; and a needle with a capturing mechanism for capturing and retaining the single tail, wherein contact of the needle with the single tail allows the single tail to move in a direction about parallel to a longitudinal axis of the needle and of the shaft. A movable jaw (movable member or upper jaw) is configured to pivot towards the tip and provided with a securing mechanism for secure placement of a loop of the flexible construct.

Another exemplary suture passer instrument comprises: a Shaft having a longitudinal axis, a distal end, and a proximal end; a stationary jaw (stationary member or tip) at the distal end of the shaft, the stationary jaw having an opening for a needle to pass through and a securing mechanism for securely holding a suture tail of a suturing construct, and then loading the suture tail into a hook of the needle when the needle passes through the opening; a movable jaw (movable member) configured to pivot towards the stationary jaw and having a securing mechanism for securely holding a suture loop of the suturing construct; a needle with a hook for retaining the single suture tail; and an actuator configured to move the movable jaw with respect the stationary jaw.

Another embodiment of a suture passer instrument includes: a hollow shaft having a longitudinal axis, a distal end, and a proximal end; a mounted distal jaw fixedly mounted to the shaft at the distal end of the shaft; a movable jaw (movable member) configured to pivot towards the mounted distal jaw to clamp tissue between the mounted distal jaw and the movable jaw, and to retract from the distal jaw to release tissue clamped between the mounted distal jaw and the movable jaw; a needle configured to slidably extend through tissue being clamped between the movable jaw and the mounted distal jaw, where the needle can extend independently of the movable jaw, the needle having a pointed end for piercing through tissue, and a hook configured to retain suture while the needle is pulled back through the tissue; the mounted distal jaw having an opening for the needle to pass through, and a securing mechanism to securely hold a single tail of a suturing construct and then load the single tail into the hook of the needle when the needle passes through the opening; the moving jaw having a securing mechanism to securely hold a loop of the suturing construct; and a handle configured to move the moving jaw with respect to the mounted distal jaw.

Methods of passing sutures are also disclosed. A first portion of a flexible construct is passed through tissue and a second portion of the flexible construct is positioned around the tissue, in one step. The first portion is passed through the second portion and then through the tissue. The flexible construct may be a suture loop terminating in a single tail. The single tail is passed through the suture loop and through the tissue.

Methods of suturing tissue are also disclosed. In an embodiment, tissue can be sutured with an instrument by securing a flexible strand within a tip of a suture passing instrument; capturing a single tail of the flexible strand with a needle; and passing the single tail of the flexible strand through a continuous, uninterrupted loop of the flexible strand and through the tissue.

An exemplary method of suturing tissue comprises: (i) clamping, securing, or holding at least a portion of a tissue with a suture passing instrument; (ii) securing a flexible construct formed of a flexible strand with a loop terminating in a single tail within a tip of the instrument; (iii) capturing the single tail with a needle; and (iv) pulling back the needle to pass the single tail through the loop and the clamped tissue.

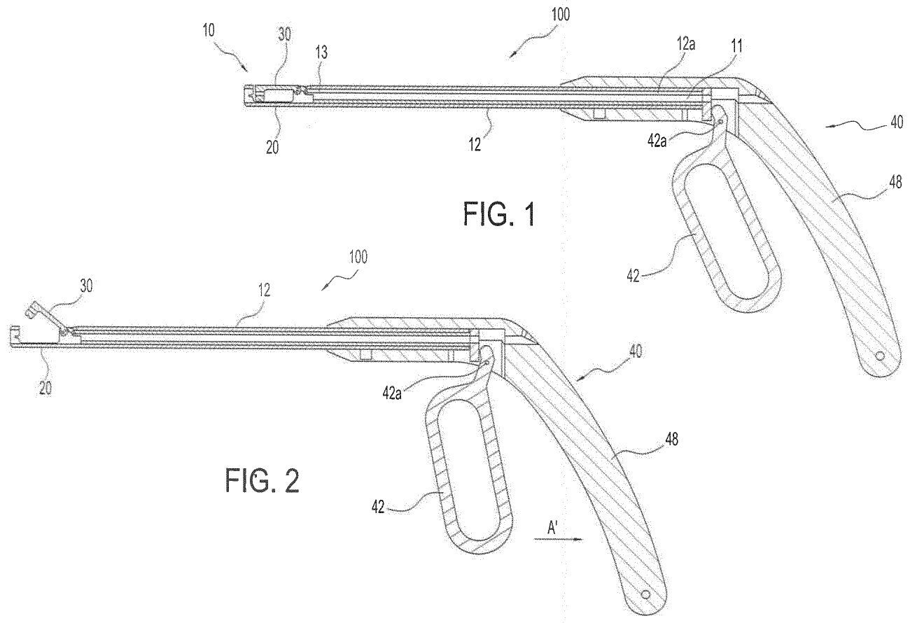

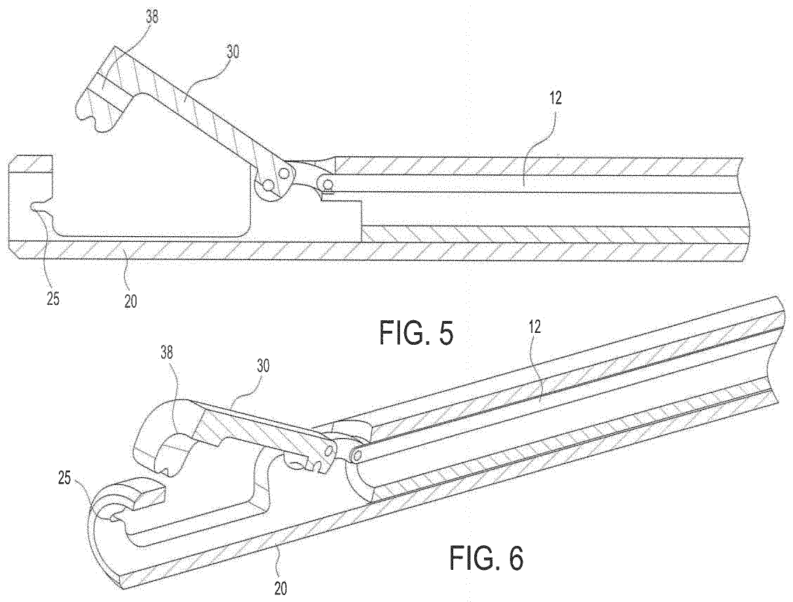

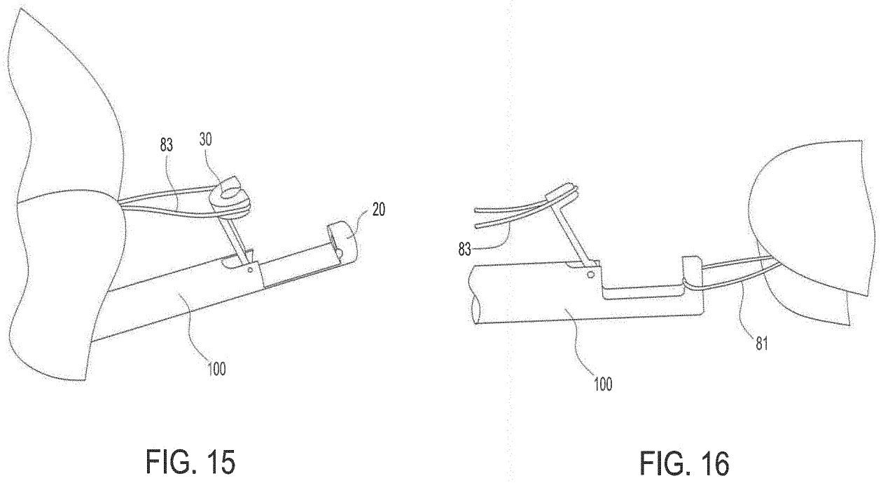

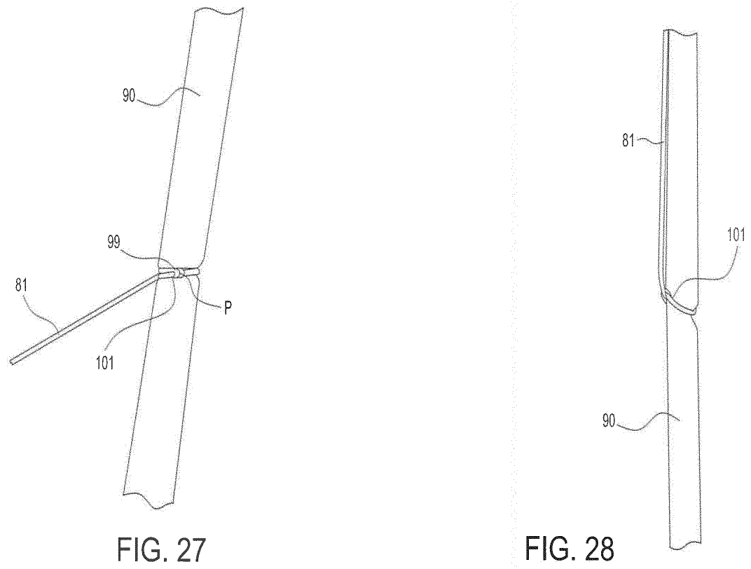

Referring now to the drawings, where like elements are designated by like reference numerals, FIGS. 1-14 illustrate various structural elements of suture passing instrument 100, 200 of the present disclosure provided with a tip configured to allow a flexible construct formed of a flexible strand having a loop terminating in a single tail to be loaded thereon and a handle assembly allowing the two components (loop and single tail) to be passed independently (and without the need to remove the instrument from the surgical site to reload it with an additional strand). FIGS. 15-28 illustrate an exemplary method of tissue repair with the suture passing instrument 100, 200.

As shown in FIGS. 1-6, suture passing instrument 100 (suture passer 100; instrument 100; surgical instrument 100 comprises an elongated tubular member or shaft 12 having a longitudinal axis 12a, a proximal end 11 a distal end 13 and an axial throughbore therein. Shaft 12 may he a tube or a narrow-diameter rod of dimensions that permit the tubular member to he introduced through an associated cannula (for example, an 8.25 cannula) in a minimally invasive procedure, such as arthroscopic or other endoscopic procedures, or into a body cavity, such as the abdominal cavity. Alternatively, shaft 12 may be an open U channel (and not a closed tube).

Elongated tubular member 12 connects, and extends between, a handle assembly 40 and a tip 10 consisting of a pair of jaws comprising a distal jaw 20 (first jaw 20, or stationary jaw 20) and a proximal jaw 30 (second jaw 30, movable jaw 30, movable member 30, or clamp 30).

Distal jaw 20 is provided at the distal end 13 of the instrument and is preferably integral with (fixedly mounted to) the tubular member 12. Proximal jaw 30 is movable with respect to the distal jaw 20 and pivots with respect to it. As illustrated in FIGS. 1-5, distal jaw 20 is substantially straight with respect to the shaft, while the proximal jaw 30 may be either straight or curved, and may be provided with a plurality of teeth to enhance tissue grasping and suture passing stability.

Distal jaw 20 of instrument 100 includes a securing mechanism 25 provided at the distal end of the jaw 20 that is configured to releasably support a length of flexible strand 81 therein. The securing mechanism 25 is formed integrally with the jaw 20 and includes an opening or slot 25 which may be a transversal suture loading slot 25. Slot 25 can be arcuate, to allow suture or a strand of flexible material to extend therethrough, in a position about perpendicular to the longitudinal axis of the elongated tubular member. Slot 25 may include a transversal channel with two lateral slots, grooves or wedges, each of the lateral slots, grooves or wedges being disposed on each end of the transversal slot, to allow increased suture positioning for facture suture manipulation with the needle construct.

In an embodiment, the securing mechanism 25 is designed to allow secure placement of the flexible strand 81 within it, while allowing a needle 55 to travel in a to- and fro-direction relative to the transversal flexible strand. In this manner, when needle 55 is advanced to travel toward the distal jaw 20, to capture the flexible strand 81, the needle can pass past the transversal slot to allow capturing of the flexible strand in notch 56 of hook 55a, and then pull back in an opposite direction (again passing past the transversal slot) to allow pulling of the flexible strand 81 away from the distal end of the instrument.

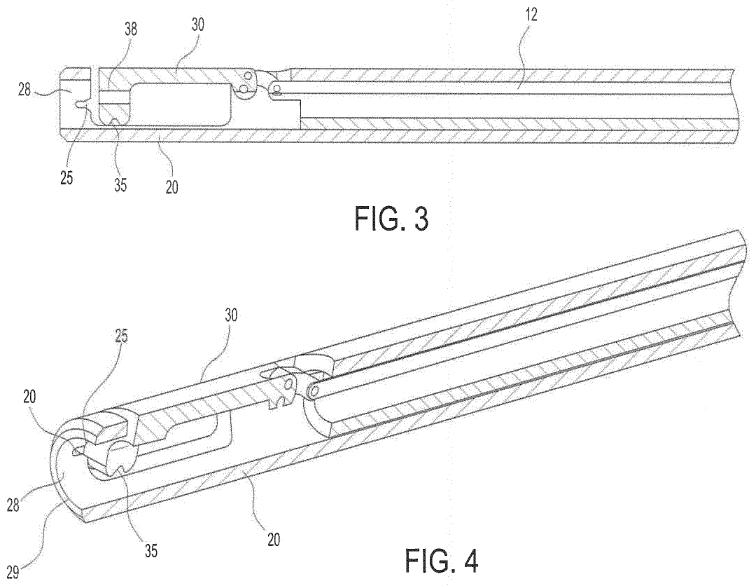

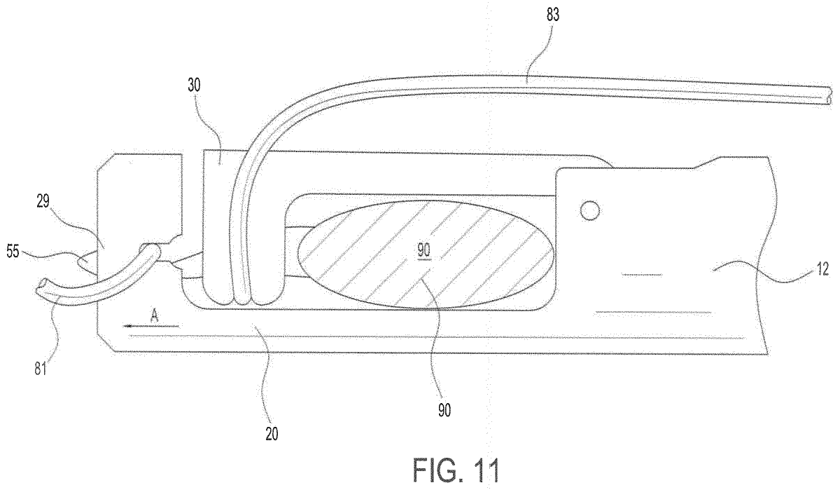

Distal jaw 20 is also provided with a needle passage 28 and an opening 29 that allows needle 55 to pass and extend therethrough, as more clearly illustrated in FIGS. 3 and 11, for example. Opening 29 (FIG. 11) is provided in a most distal end of jaw 20 and communicates with the needle passage 28. Preferably, the opening 29 has the shape of a channel with a rectangular or square cross-section to allow the needle (which may have a generally square or rectangular cross-section, or a generally circular cross-section) to pass therethrough. As shown in FIG. 11, distal opening 29 allows needle 55 to exit in a direction about parallel to the longitudinal axis of the instrument.

In an exemplary embodiment, needle 55 also passes through a channel or needle passage 38 (FIG. 33 of the proximal jaw 30 (movable jaw 30) when the proximal jaw 30 is in the operative, piercing, deployed or closed position. Needle 55 is substantially disposed within the shaft 12 when the needle is in a non-operative, non-piercing, or non-deployed position. In an operative position, and as detailed below, the needle is advanced distally to pass through the passage 38 of the proximal jaw 30 and through the passage 28 of the distal jaw, to exit opening 29 in the distal law 20.

Preferably, needle 55 has a narrow, pointed tip at a distal end and a hook 55a that is adjacent the pointed tip and that has an opening (notch) 56 facing the proximal end of the instrument (FIG. 8). The opening 56 of hook 55a also preferably faces downwardly (i.e., in a plane extending below the longitudinal axis 12a of the instrument). Needle 55 may preferably be formed of nitinol or other suitable material.

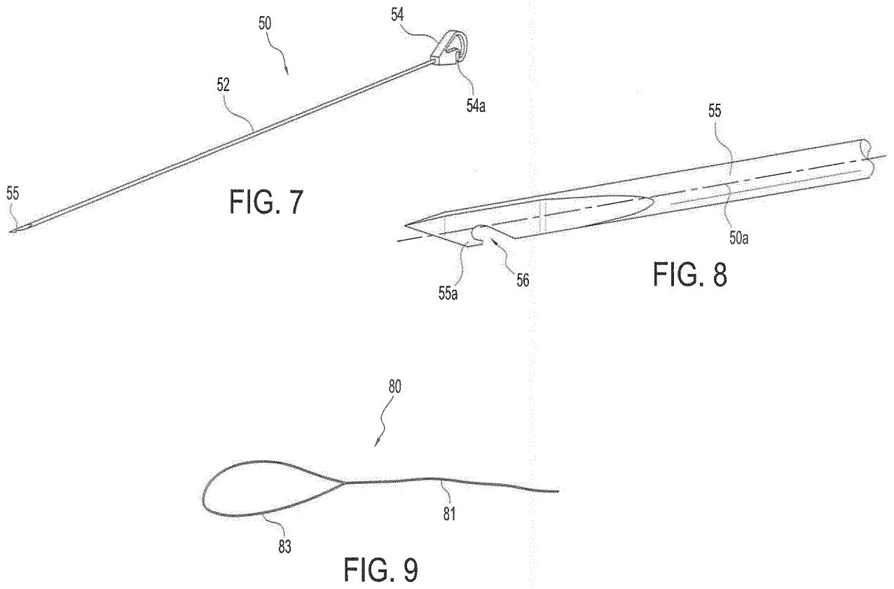

Needle actuator 50 (FIG. 7) houses needle 55 and comprises shaft 52 (which is slidingly received in the axial throughbore (channel) of the elongated tubular member 12) and a most proximal end 54 provided with a slot that is engaged securely into a cross pin 54a on the proximal end of handle 40 of the suture passing instrument (FIG. 1). The needle actuator facilitates passing the flexible strand 81 through the tissue. In use, needle actuator 50 is actuated relative to the longitudinal axis 12a of the tubular member, and needle 55 is pushed forward toward opening 29 to exit the opening as shown in FIG. 11, for example.

Proximal jaw 30 forms part of the tip 10 and may pivot with respect to the tip or distal jaw 20. The proximal jaw 30 includes longitudinal channel 38 and a securing mechanism 35 provided at the distal end of the proximal jaw 30. In exemplary embodiments, the securing mechanism 35 may be a suture loading slot 35 (tooth 35). Slot 35 can be arcuate, to allow suture or a strand of flexible material to extend therethrough, in a position about perpendicular to the longitudinal axis of the elongated tubular member. Slot 35 may include a transversal channel with two lateral slots, grooves or wedges, each of the lateral slots, grooves or wedges being disposed on each end of the transversal slot, to allow increased suture positioning for future suture manipulation with the needle construct. The longitudinal channel and the securing mechanism (loading slot) are configured to allow at least one flexible loop 83 (a continuous, flexible, uninterrupted loop 83 of flexible material 80) to extend therethrough and to be securely positioned within the securing mechanism (loading slot). Slot 35 may have various configurations, for example, a groove (such as a V groove) or a wedge, to allow suture positioning for future suture manipulation with the instrument 100. The securing mechanism 35 is formed integrally with the proximal jaw 30.

FIG. 9 illustrates an exemplary flexible strand 80 (flexible material 80, suture construct 80, suturing construct 80, single tail/loop construct 80) to be loaded onto the tip 10 of the instrument. Flexible strand 80 has a first region 81 and a second region 83. In an embodiment, the first region 81 is a single end or single tail 81, and the second region 83 is a continuous, uninterrupted, flexible loop 83.

Flexible strand 80 is formed of a single strand of material that is looped joined to form loop 83 and single tail 81. In an embodiment, flexible strand 80 has a length and two ends that form a loop 83 terminating in a single tail 81. In an embodiment, flexible strand 80 has one end that forms loop 83 and the other end forming single tail 81 (flexible end 81). Loop 83 may be a continuous, uninterrupted, flexible loop formed of a material such as suture. Loop 83 terminates in single flexible end or tail 81. In one embodiment, the perimeter of loop 83 may be fixed. In one embodiment, loop 83 and tail 81 may be formed of suture having a round cross-section. The suture may have the same or different diameters. Loop 83 may be formed by splicing the flexible strand through itself, or by other methods known in the art, such as fusion, gluing, bonding, joining, braiding, interlinking, etc.

Flexible strand 80 (suturing construct 80) may include a single filament, or fiber, or can include multiple continuous filaments, segments or regions of filaments that have different configurations (for example, different diameters and/or different compositions). The filament regions/segments may each be homogenous (i.e., formed of a same material) or may be a combination of homogenous and heterogenous (i.e., formed of a plurality of materials). Exemplary materials may include suture, silk, cotton, nylon, polypropylene, polyethylene, ultrahigh molecular weight polyethylene (UHMWPE), polyethylene terephthalate PET), and polyesters and copolymers thereof, or combinations thereof.

In an embodiment, the flexible strand 80 is made of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The flexible strand may be also formed of suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein.

Flexible strand 80 may also include, and be manufactured with, any kind of material (suture, nylon, silk, UHMWPE. metal, bioabsorbable, etc.) that can allow the flexible strand to form a loop terminating in a single tail.

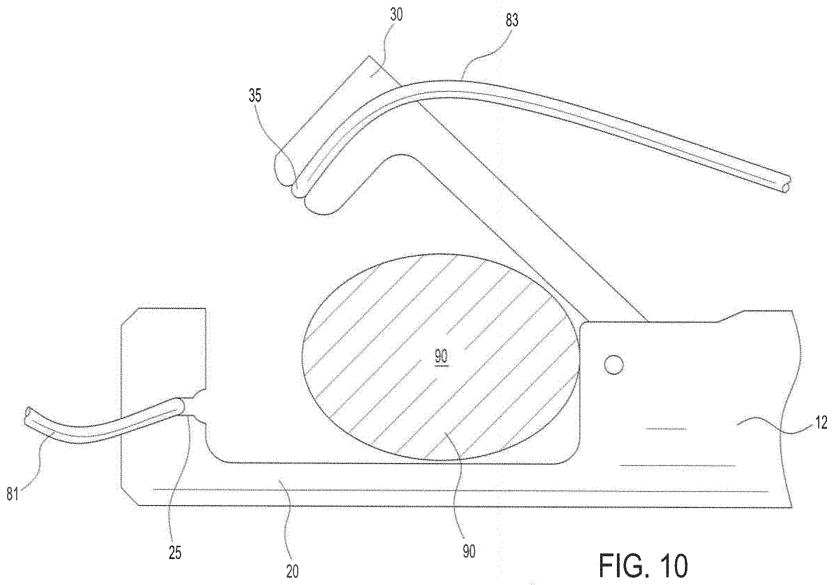

In an exemplary embodiment, flexible strand 80 is loaded onto the instrument 100 as shown in FIG. 10, i.e., with loop 83 loaded onto securing mechanism 35 (loading slot) through the longitudinal channel of the proximal jaw 30, and with the single tail 81 loaded through the longitudinal channel and into slot 25 of the distal jaw 20 for further engagement by needle 55.

Handle assembly 40 of the suturing instrument 100 (illustrated in FIGS. 1 and 2) is provided at the proximal end 11 of the instrument and includes a trigger mechanism 42 (or actuating mechanism 42) and a handle 48, the trigger mechanism 42 being pivotally connected to the handle 46. The trigger mechanism 42 may consist of a finger lever 42 which, when actuated, is designed to move/pivot the proximal jaw 30 (movable member 30) relative to the distal jaw 20. The finger lever 42 is designed to move from a first position to a second position, with a spring 42a provided to maintain tension on the finger lever 42 and to force the finger lever 42 to go back to a default position. At its default position, the finger lever 42 is closest from the distal end of the handle 28.

instrument 100 further includes an actuator or a second trigger mechanism (not shown), which is provided within the shaft 12 and at the distal end of the shaft The actuator is connected to the finger lever 42 and is designed to pivot the movable jaw 30 from a first position to a second position as the finger lever 42 is moved from a first position to a second position, and to clamp or secure tissue 90 between the movable jaw 30 and the tip or stationary jaw 20.

FIGS. 10-13 illustrate schematic steps of a method of tissue repair (e.g., tendon or ligament repair) with instrument 100 and exemplary flexible strand 80.

FIG. 10: suture loop 83 is pulled into slot 35 of proximal jaw 30; single tail 81 is placed into slot 25 of distal jaw 20. Instrument 100 loaded with flexible strand 80 is advanced to hold tissue 90 securely; tissue 90 is clamped or secured between the pair of jaws 20, 30 (i.e., tip 20 and movable jaw 30).

FIG. 11: proximal jaw 30 pivots to the operative position; needle 55 is advanced to penetrate tissue 90 and pass through channel 38 of proximal jaw 30 and through channel 28 of distal jaw 20; needle 55 is advanced in the direction of arrow A.

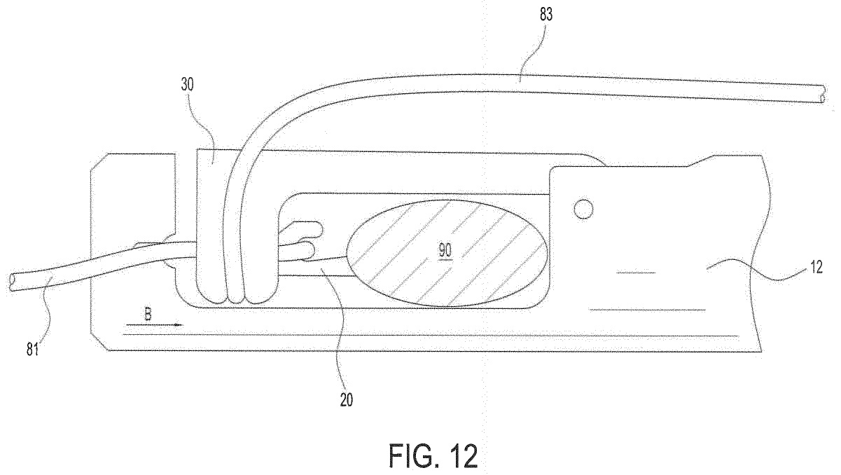

FIG. 12: when the needle hook 55a passes the suture tail 81, the tail 81 is pushed into the needle notch 56; the needle 55 is pulled back, retrieving suture 81 from tip 20 and through the loop 83; needle 55 is pulled back in the direction of arrow B.

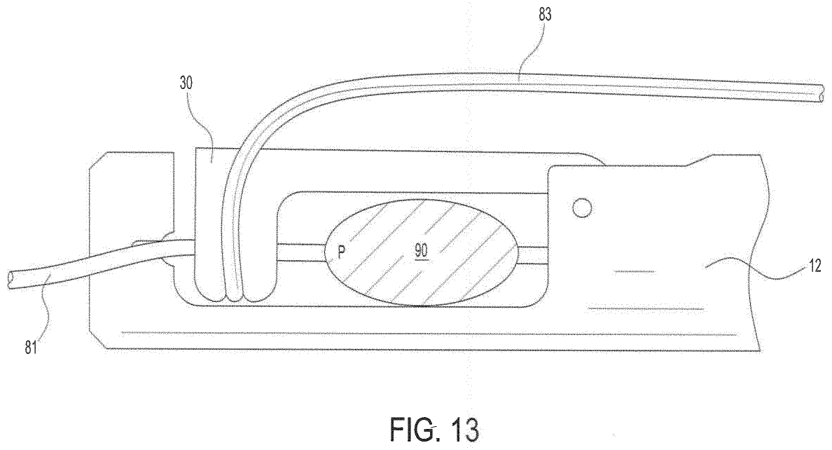

FIG. 13: the needle 55 pulls suture tail 81 back through the tissue 90 penetrating tissue 90 at location or point “P”; advance the needle 55 and pull down on single tail 81 to release; loop 83 is pulled by pulling, the single tail 81 to be positioned around tissue 90 to complete a Loop ‘N’ Tack™ stitch in one step.

Soft tissue 90 is luggage-tagged with looped suture 83 (or suture tape 83) while—and after—the soft tissue is pierced with the free end 81 of the suture 80. A Loop ‘N’ Tack™ knotless tenodesis technique is an all-arthroscopic technique using a SutureTape FiberLink™ suture 80. The biceps is “luggage tagged” with the loop 83 of the FiberLink™ suture 80 and the free end 81 of the FiberLink™ suture 80 which pierces the biceps 90 and passes through it. A fixation device (for example, a SwiveLock® or PushLock® anchor) or similar structures may be employed to anchor the free suture end 81 and the biceps 90 intra-articularly at the top of the bicipital groove.

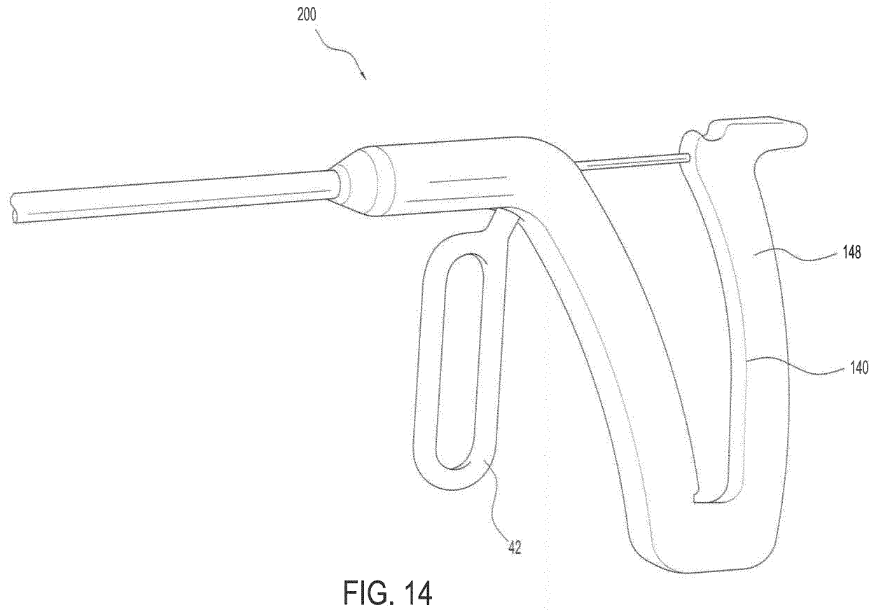

FIG. 14 illustrates suture passing instrument 200 (suture passer 200; instrument 200; surgical instrument 200) Which is about similar to suture passing instrument 100 (detailed above) but differs from it in the shape and configuration of handle 148 of handle assembly 140. Handle 148 comprises a proximal end and a distal end that could be actuated and brought together. The proximal and distal ends of the handle 148 may be connected via a link (not shown) and tension between the proximal and distal ends may be additionally maintained using springs. By actuating the hand mechanism 148 (i.e., by bringing together the proximal and distal ends of the handle 148), pusher 54 of the needle actuator 50 advances needle 55 to capture the single tail or end 81 of flexible strand 80 positioned within slot 25, and to further pull back the flexible strand 81 (captured in notch 56 of hook 55a of the needle) through loop 83 and through tissue 90 to be sutured.

Instrument 100, 200 is a one-step instrument that allows a loop 83 of a flexible construct 80 to be positioned around tendon 90 (surrounding the tendon) while passing/driving a distal end 81 of the suture loop construct 80 through the tendon 90. The novel instrument eliminates multiple instruments needed to perform a Loop ‘N’ Tack™ stitch, improving OR time and the efficiency of the overall procedure.

The instrument allows surgeons to conduct a Loop ‘N’ Tack™ stitch on a tendon with improved efficiency and by eliminating multiple instruments necessary in open or arthroscopic procedures. The instrument 100 includes a shaft (movable jaw 30) with a tooth 35 that captures the suture loop 83. The free end 81 of suture 80 is captured by the end (distal jaw 20) of the shaft.

FIGS. 15-28 illustrate an exemplary method of tissue repair 101 with exemplary suture passing instrument 100, 200. Repair 101 is a Loop ‘N’ Tack™ stitch repair. A method of employing instrument 100, 200 begins by loading the needle 55 into the cannulation on the proximal end of the shaft 12 of instrument 100. The slot on the proximal end 54 of the needle actuator 50 is engaged securely into cross pin 54a on the proximal end of handle 28 of the instrument.

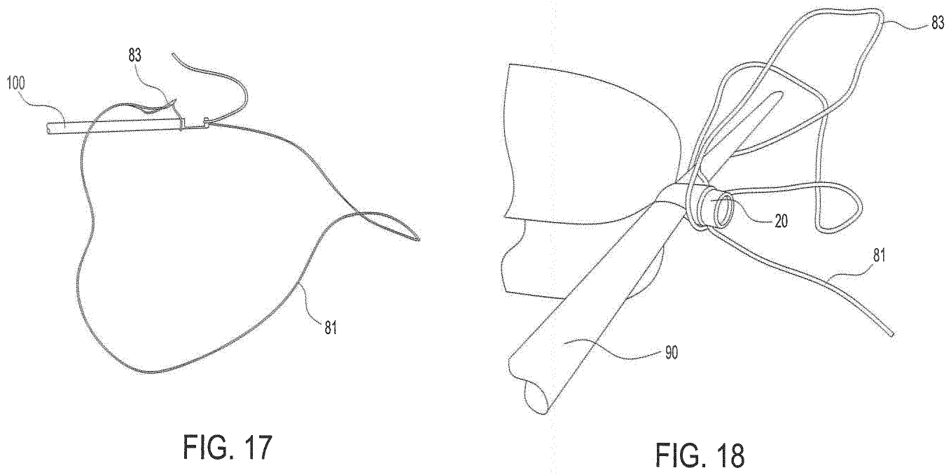

Flexible strand 80 is loaded onto the instrument, as shown in FIGS. 15-17. Loop 83 is positioned onto securing mechanism 35 of the proximal jaw 30 while single end 81 is loaded onto securing mechanism 25 (transversal slot 25) on the distal jaw 20 of the instrument 100. FIG. 17 shows both the loop 83 and single tail 81 (free end 81) secured onto instrument 100 with the proximal jaw 30 open.

Reference is now made to FIG. 18. Using the finger lever 42 on the suture passing instrument 100 (i.e., actuating the finger lever 42 in the direction of arrow A′ of FIG. 2), the proximal jaw 30 pivots towards the distal jaw 10 and tissue 90 is clamped between the two jaws 20, 30. With the jaws closed, loop 83 is located proximally to the single end 81.

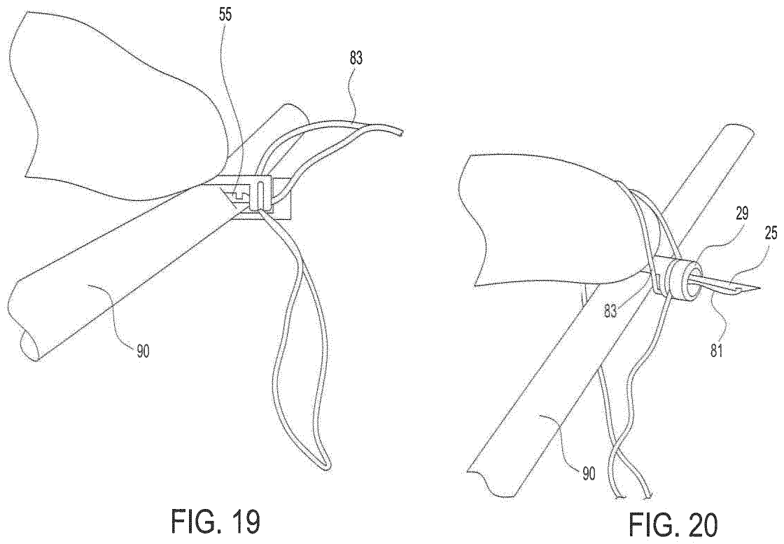

With the tissue 90 clamped between the jaws and the sutures positioned around tissue 90, needle 55 is advanced distally (FIG. 19) such that the needle exits the opening 29 on the distal jaw 20 (FIG. 20). The pointed tip of the needle 55 pierces completely through the tissue (e.g., tendon or articular capsule), passes through channel 38 of proximal jaw 30 (loaded with loop 83) and through the loop 83, and then the hook 55a on the needle 55 captures the tail 81 (loaded in the slot 25 of the distal jaw 20), as shown in FIG. 20.

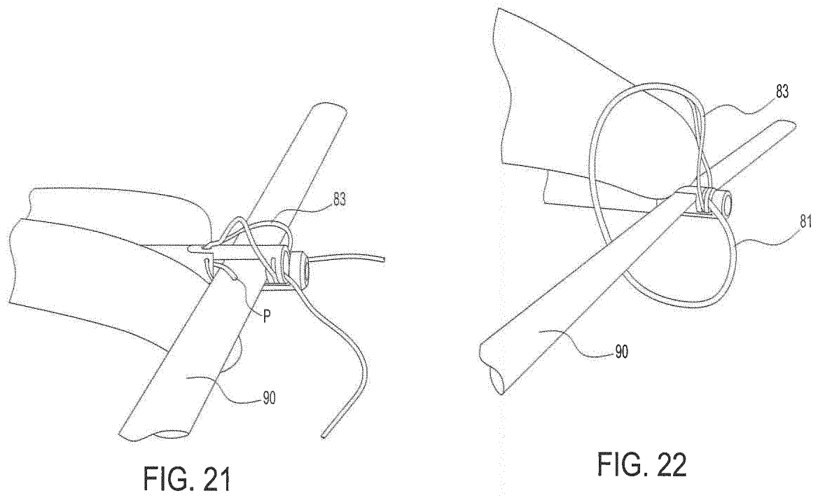

By releasing the distal and proximal ends of handle 148 (for example), the needle 55 is pulled back (FIG. 21) and the suture 81 captured in hook 55a and notch 56 of the needle is pulled through the loop 83 and then pierces tissue 90 at a location “T” (FIG. 21).

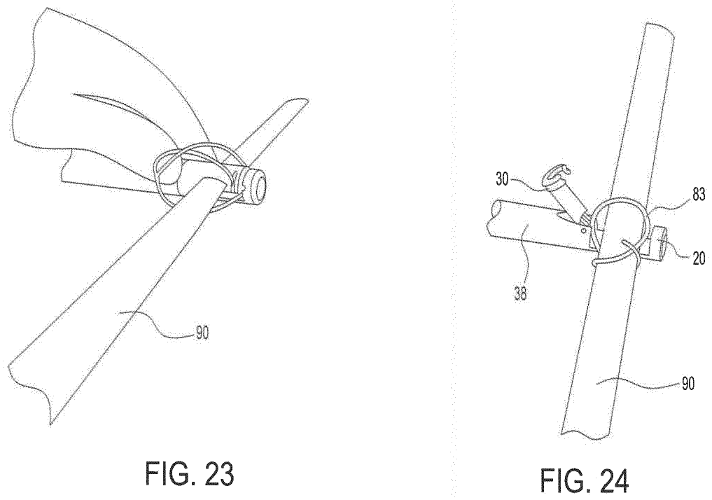

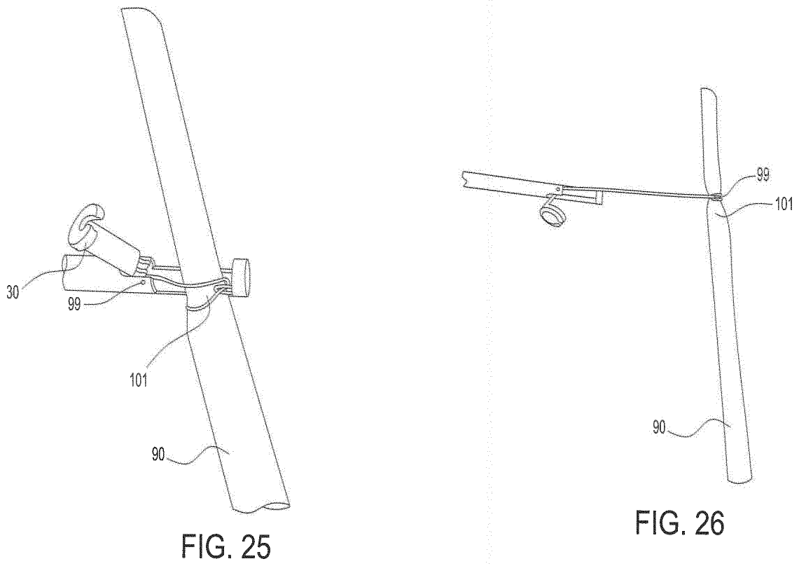

Free end 81 of the suture construct 80 is pulled back through the soft tissue 90 in a direction about parallel to longitudinal axis 12a of the shaft 12 of the instrument, pulling, the loop 83 closer to tissue 90 (FIGS. 22 and 23). By releasing, the finger lever 42, the proximal jaw 30 pivots to the open position (pulls away from the distal law 20) and the tissue 90 is released, as shown in FIG. 24. FIGS. 25 and 26 illustrate formation of loop 99 around tissue 90 and adjacent location “P” where the free end 81 pierces the tissue 90. Pulling on the free end 81 tightens loop 99 around tissue 90 to complete repair 101 or luggage tag stitch 101 (FIGS. 26-28).

A suture passer as disclosed herein may be employed in various surgical medical procedures for advancing a suture in proximity of a surgical site. A suture passer as disclosed herein may also be employed with a suture and a cannulated instrument during such surgical procedures. For example, instrument 100, 200 may be employed in endoscopic surgery. The term “endoscopic surgery” refers to surgical procedures within a patient's body through small openings as opposed to conventional open surgery through large incisions. Additionally, a suture passer as disclosed herein may be utilized in other general surgical and specialty procedures that require suturing. A suture passer as disclosed herein may be also used in repairs where suture visibility or finger access can be limited.

It will be appreciated, of course, that While a suture passer as disclosed herein may be particularly useful for performing remote procedures through access sheaths, trocars and cannulated surgical instruments, a suture passer as disclosed herein can also be used in open surgical procedures.

In an illustrative embodiment, a method of suturing tissue using, a suture passer 100, 200 comprises: (i) engaging a tail 81 of a flexible strand 80 within a slot 25 of a securing mechanism 25 at the distal end 20 of the instrument; (ii) engaging a continuous, uninterrupted, flexible loop 83 of the flexible strand 80 within a slot 35 of a securing mechanism 35 at a proximal jaw 30 of the instrument; (iii) advancing a needle 55 through tissue and through passages thrilled in jaws 20, 30 of the instrument; and (iv) subsequently, capturing tail 81 of the flexible strand 80 with the needle 55 and advancing the tail 81 in a direction about parallel to the longitudinal axis 12a of the instrument, and through the continuous, uninterrupted, flexible, loop 83 and through tissue 90.

According to another exemplary and illustrative embodiment only, a method of suturing a tissue using the suture passing instrument 100, 200 comprises: (i) loading a needle 55 onto the instrument; (ii) loading a suture 80 formed of a suture loop 83 terminating in a single tail 81 into slots formed in jaws 20, 30 of the instrument; (iii) bringing a proximal jaw 30 towards the distal jaw 20 by actuating a finger lever 42, so that the proximal jaw 30 and the distal jaw 20 clamp tissue 90 therebetween; (iv) actuating a handle 48 to advance the needle 55 into a first direction (towards the tail loaded onto the slot of the distal jaw); (v) pulling the needle 55 in a second direction which is opposite the first direction, and capturing the tail 81 of the suture 80 with a hook 56 and notch 56a of the needle 55; and (vi) pulling the tail 81 through the suture loop 83 and through tissue 90 in a direction about parallel to the longitudinal axis 12a of the instrument.

A suture passing instrument 100, 200 comprises: a shaft 12 having a longitudinal axis 12a, a distal end 13, and a proximal end 11; a first jaw 20 fixedly mounted to the distal end of the shaft 12, the first jaw 20 having a passage 28 for receiving a needle 55, and a distal opening 29 to allow the needle 55 to pass therethrough; a second jaw 30 being movable with respect to the first jaw 20, the second jaw 30 having a passage 38 for receiving needle 55; and a handle assembly 40 configured to move the needle 55 from a first position to a second position, and to move the second jaw 30 with respect to the first jaw 20, wherein the first jaw 20 comprises a securing mechanism 25 to secure a single end 81 of a flexible strand 80 at its most distal end, and wherein the second jaw 30 comprises a securing mechanism 35 to secure a continuous, uninterrupted, flexible loop 83 terminating in end 81 of the flexible strand 80.

According to another exemplary and illustrative embodiment only, a method of suturing a tissue using the suture passing instrument 100, 200 of the present disclosure comprises the steps of: (i) loading a first portion 81 of a suture strand 80 onto a bottom jaw 20 of a suture passing instrument 100, 200; (ii) loading a second portion 83 of the same suture strand 80 onto a top jaw 30 of the suture passing instrument; (iii) closing the jaws 20, 30 on a desired, first region of the tissue 90 using a finger lever and advancing a needle 55 through the first region of the tissue 90 at a location P; (iv) capturing the first portion 81 of the suture strand 80 with the needle 55; (v) retracting the needle 55 with the captured first portion 81 through the second portion 83 of the same suture strand 80 and through the first region on the tissue 90; and (vi) securing the second portion 83 of the same suture strand around a second region of the tissue 90 to form a loop 99 around the second region.

The device 100, 200 is first loaded with the required suture 80 which then allows the user to encompass the soft tissue 90 with the suture 80. Once the jaw 30 closes around tissue 90, free end 81 and loop 83 of suture 80 are positioned around tissue 90 (even if suture 80 is not tightened around tissue 90). Once the tissue 90 is captured and the sutures 81, 83 are properly positioned to encapsulate the tissue, the needle 55 can be deployed to penetrate the tissue 90. When the needle 55 is retracted, it retrieves the free end 81 of suture and pulls it through the loop 83 and through the tissue 90. The free end 81 passes through both the loop 83 and through the tissue 90. The device (instrument) 100, 200 is removed and the free end 81 of the suture (passed through the loop and through the tissue) is tightened to produce the stitch 101 around and through tissue 90. When the user reduces slack, what is produced is the Loop ‘N’ Tack™ (a luggage tag stitch) 101 around and through soft tissue 90.

Flexible strand 80 may be in the form of any elongated members, fibers, or materials, or combinations thereof. Flexible strand 80 can include a single filament, or fiber, or can include multiple continuous filaments, segments or regions of filaments that have different configurations (for example, different diameters and/or different compositions). The filament regions/segments may each be homogenous (i.e., formed of a same material or may be a combination of homogenous and heterogenous (i.e., formed of a plurality of materials). Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof.

Flexible strand 80 may be formed of suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein. In an embodiment, flexible strand 80 may be made of fibers of ultrahigh molecular weight polyethylene (UHMWPE) or the FiberWire® suture (disclosed in U.S. Pat. No. 6,716,234, the disclosure of which is hereby incorporated by reference in its entirety herein). High strength suture such as FiberWiret suture (Arthrex), FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneemat (DSM N.V., Heerlen, the Netherlands), braided with at least one other fiber, natural or synthetic, to form lengths of suture material.

Flexible strand 80 may be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture, loop security, pliability, handleability or abrasion resistance, for example.

Flexible strand 80 may be also provided with tinted tracing strands, or otherwise contrast visually with other areas/regions of the construct, which remains a plain, solid color, or displays a different tracing pattern, for example. Various structural elements of flexible strand 80 such as loop 83 and/or tail 81 may be visually coded, making identification and handling of the suture loops and ends simpler. Easy identification of suture in situ is advantageous in surgical procedures.

The term “high strength suture” is defined as any elongated flexible member, the choice of material and size being dependent upon the particular application. For the purposes of illustration and without limitation the term “suture” as used herein may be a cable, filament, thread, wire, fabric, or any other flexible member suitable for tissue fixation in the body.

The term “luggage tag stitch” is defined as any cinch or suture loop that is formed by the luggage tag technique.

What is claimed is:

1. A suture passing instrument comprising:
a shaft having a cannulation, a longitudinal axis, a distal end, and a proximal end;
a needle passing through the cannulation of the shaft;
a first jaw and a second jaw, the first jaw fixedly mounted to the distal end of the shaft, the first jaw having a first passage for receiving the needle, and a distal opening to allow the needle to pass therethrough; the second jaw being movable with respect to the first jaw, the second jaw having a second passage for receiving the needle; and
a handle assembly configured to move the needle from a first position to a second position, and to move the second jaw with respect to the first jaw,
wherein the first jaw comprises a first securing mechanism to secure a first portion of a flexible strand, and wherein the second jaw comprises a second securing mechanism to secure a second portion of the flexible strand.

2. The suture passing instrument of claim 1, wherein the flexible strand is a suture loop terminating in a single tail and wherein the first portion is the single tail and the second portion is the suture loop.

3. The suture passing instrument of claim 1, wherein the handle assembly comprises a handle and a trigger mechanism, wherein the handle comprises a proximal member and a distal member, and
wherein the trigger mechanism comprises a finger lever being pivotally connected to the distal member of the handle.

4. The suture passing instrument of claim 1, wherein the needle is provided with a sharp tip, a hook, and a notch.

5. The suture passing instrument of claim 1, wherein the second jaw is a pivotably movable member.

6. The suture passing instrument of claim 1, wherein the tissue is soft tissue.

7. The suture passing instrument of claim 1, wherein the tissue is tendon or ligament.

8. The suture passing instrument of claim 1, wherein the flexible strand is a braided suture comprising a plurality of high strength fibers.

9. A method of suturing tissue with a suture passing instrument in recited in claim 1 comprising:
advancing the needle in a first direction through the second passage and through the first passage;
capturing the first portion of the flexible strand with the needle: and
advancing the needle in a second direction which is opposite the first direction to pass the first portion through the second portion and through the tissue.

10. The method of claim 9, wherein the first portion is a suture tail and the second portion is a suture loop terminating in the suture tail.

11. The method of claim 9, further comprising the steps:
piercing the tissue with the first portion; and
pulling on the first portion to position the second portion around the tissue and forming a loop around the tissue.

12. The method of claim 9, wherein the tissue is tendon or ligament.

13. A method of forming a luggage tag stitch on tissue, comprising:
securing a flexible end of a suturing construct on a first jaw of a suture passer, and securing a loop of the suturing construct on a second jaw of the suture passer;
advancing, in a first direction, a needle of the suture passer to capture the flexible end;
advancing, in a second direction, the needle with the captured flexible end to pass the flexible end through the loop and to pass the flexible end through the tissue; and
pulling on the flexible end to position the loop around the tissue to form a luggage tag stitch.

14. The method of claim 13, wherein the suturing construct is a single strand with the loop terminating in the flexible end.

15. The method of claim 13, wherein the first direction is opposite the first direction.

16. The method of claim 13, wherein the suturing construct is formed of suture, silk, cotton, nylon, polypropylene, polyethylene, ultrahigh molecular weight polyethylene (UHMWPE), polyethylene terephthalate (PET), polyesters or copolymers, or combinations thereof.

17. The method of claim 13, wherein the loop is a flexible,
uninterrupted, continuous loop having a fixed perimeter.

18. The method of claim 13, wherein the tissue is soft tissue.
